(19) United States
(12) Patent Application Publication
(10) Pub. No.: US 2026/0083406 A1
(43) Pub. Date: Mar. 26, 2026

(54) INFORMATION PROCESSING DEVICE, MONITORING DEVICE, AND NON-TRANSITORY COMPUTER-READABLE MEDIUM

(71) Applicant: NIHON KOHDEN CORPORATION, Tokyo (JP)

(72) Inventor: Tetsuri ARIYAMA, Saitama (JP)

(73) Assignee: NIHON KOHDEN CORPORATION, Tokyo (JP)

(21) Appl. No.: 19/325,017

(22) Filed: Sep. 10, 2025

(30) Foreign Application Priority Data
Sep. 20, 2024 (JP) 2024-163955

(51) Int. Cl. A61B 5/00; A61B 5/08

(52) U.S. Cl. CPC A61B 5/7282; A61B 5/0816; A61B 5/7235; A61B 5/7275

(57) ABSTRACT

An interface receives a signal corresponding to a waveform of physiological information involving cyclic changes from a sensor attached to a subject. A processor causes, in response to the signal, an output device to output information corresponding to an estimated value of a respiration rate of the subject. The processor extracts multiple feature quantities corresponding to respiration of the subject from the signal included in each of multiple time slots. The processor applies frequency analysis processing with respect to each of the feature quantities to acquire multiple preliminary estimation values of the respiration rate. The processor determines the estimated value based on a weighted average value that is associated with appearance frequencies of the preliminary estimation values.

### CROSS-REFERENCE TO RELATED APPLICATION

The present application is based on Japanese Patent Application No. 2024-163955 filed on Sep. 20, 2024, the entire contents of which are incorporated herein by reference.

### BACKGROUND

The presently disclosed subject matter relates to an information processing device configured to process physiological information of a subject. The present disclosure also relates to a monitoring device configured to monitor physiological information of a subject. The presently disclosed subject matter also relates to a non-transitory computer-readable medium having stored a computer program adapted to be executed by a processor installed in the information processing device.

It is well-known a technique in which a waveform of pulse waves that is an example of a waveform of physiological information involving cyclic changes is acquired from a subject to estimate a respiration rate of the subject based on the waveform. Japanese Patent No. 3627243B discloses that body motions of a subject is detected with an acceleration sensor to improve estimation accuracy of the respiration rate. Japanese Patent No. 6355152B discloses that the number of sensors for acquiring the waveform of the pulse waves to improve estimation accuracy of the respiration rate.

### SUMMARY

It is required to suppress deterioration of the estimation accuracy of the respiratory rate of the subject without increasing the number of sensors.

An illustrative aspect of the presently disclosed subject matter may provide an information processing device configured to process physiological information of a subject, comprising:
an interface configured to receive a signal corresponding to a waveform of physiological information involving cyclic changes from a sensor attached to the subject; and
a processor configured to cause, in response to the signal, an output device to output information corresponding to an estimated value of a respiration rate of the subject,
wherein the processor is configured to:
   extract multiple feature quantities corresponding to respiration of the subject from the signal included in each of multiple time slots;
   apply frequency analysis processing with respect to each of the feature quantities to acquire multiple preliminary estimation values of the respiration rate; and
   determine the estimated value based on a weighted average value that is associated with appearance frequencies of the preliminary estimation values.

An illustrative aspect of the presently disclosed subject matter may provide a monitoring device configured to monitor physiological information of a subject, comprising:
an interface configured to receive a signal corresponding to a waveform of physiological information involving cyclic changes from a sensor attached to the subject;
an output device; and
a processor configured to cause, in response to the signal, the output device to output information corresponding to an estimated value of a respiration rate of the subject,
wherein the processor is configured to:
   extract multiple feature quantities corresponding to respiration of the subject from the signal included in each of multiple time slots;
   apply frequency analysis processing with respect to each of the feature quantities to acquire multiple preliminary estimation values of the respiration rate; and
   determine the estimated value based on a weighted average value that is associated with appearance frequencies of the preliminary estimation values.

An illustrative aspect of the presently disclosed subject matter may provide a non-transitory computer-readable medium having stored a computer program adapted to be executed by a processor installed in an information processing device configured to process physiological information of a subject, the computer program being configured to, when executed, cause the information processing device to:
receive a signal corresponding to a waveform of physiological information involving cyclic changes from a sensor attached to the subject;
extract multiple feature quantities corresponding to respiration of the subject from the signal included in each of multiple time slots;
apply frequency analysis processing with respect to each of the feature quantities to acquire multiple preliminary estimation values of a respiration rate of the subject;
determine an estimated value of the respiration rate based on a weighted average value that is associated with appearance frequencies of the preliminary estimation values; and
cause an output device to output information corresponding to the estimated value.

The inventor of the present application paid attention to a fact that, when the estimated value of the respiratory rate of the subject is determined based on the multiple preliminary estimation values, one or some of them acquired with low accuracy due to some reason may adversely affect the final result of the estimated respiratory rate. According to the configuration of the above-described exemplary processing, the estimation value of the respiratory rate of the subject is determined based on the weighted average value that is calculated such that the influence of the preliminary estimation value having higher appearance frequency is enhanced. Accordingly, it is possible to suppress affection of a preliminary estimation value having lower appearance frequency that would possibly be an abnormal value. Since such processing is executed by the processor installed in the information processing device, it is possible to suppress the deterioration in the estimation accuracy of the respiratory rate of the subject without increasing the number of sensors.

### DESCRIPTION OF THE DRAWINGS

FIG. 1 illustrates a functional configuration of a monitoring device according to an exemplary embodiment.

FIG. 2 illustrates exemplary processing executed by a processor of FIG. 1.

FIG. 3 illustrates a baseline fluctuation of a waveform signal of FIG. 1.

FIG. 4 illustrates an amplitude fluctuation of the waveform signal of FIG. 1.

FIG. 5 illustrates a frequency fluctuation of the waveform signal of FIG. 1.

FIG. 6 illustrates another exemplary processing executed by the processor of FIG. 1.

FIG. 7 illustrates a specific flow of weighted averaging of FIG. 6.

FIG. 8 illustrates another exemplary processing executed by the processor of FIG. 1.

FIG. 9 illustrates another exemplary processing executed by the processor of FIG. 1.

FIG. 10 illustrates another exemplary processing executed by the processor of FIG. 1.

FIG. 11 illustrates another exemplary processing executed by the processor of FIG. 1.

### DESCRIPTION OF EMBODIMENTS

Exemplary embodiments will be described in detail with reference to the accompanying drawings.

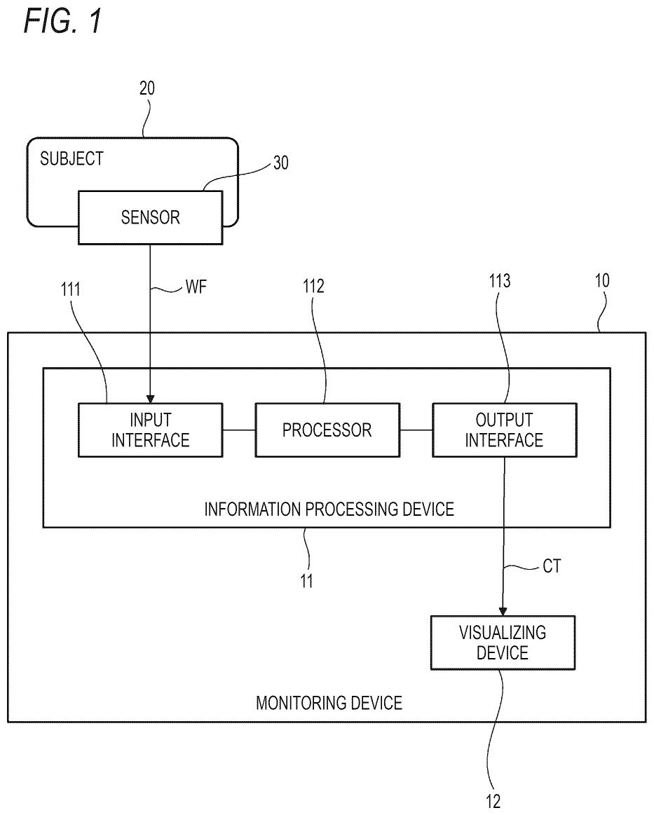

FIG. 1 illustrates a functional configuration of a monitoring device 10 according to an exemplary embodiment. The monitoring device 10 is a device configured to monitor physiological information of the subject 20. The monitoring device 10 includes an information processing device 11 and a visualizing device 12.

The information processing device 11 is a device configured to process physiological information of a subject 20. Specifically, the information processing device 11 is configured to acquire information corresponding to an estimated value of a respiratory rate of the subject 20 based on a signal corresponding to a waveform of pulse waves of the subject 20. The pulse waves is an example of physiological information involving cyclic changes. As used herein, the term “waveform” means a time series of values of specific physiological information.

The visualizing device 12 is configured to visualize information corresponding to an estimated value of the respiratory rate of the subject 20. As an example, the visualizing device 12 may be a display configured to display the information, or a projector configured to project the information. As another example, the visualizing device 12 may be a printer configured to print the information on a medium. The visualizing device 12 is an example of an output device.

The information processing device 11 includes an input interface 111. The input interface 111 is configured as a hardware interface that receives a waveform signal WF corresponding to the waveform of the pulse waves from a sensor 30 attached to the subject 20. The waveform signal WF may be an analog signal or a digital signal in accordance with the specification of the sensor 30.

Examples of the sensor 30 for acquiring the pulse waves include a photoelectric sensor including a light emitting element and a light detecting element. The light emitted from the light emitting element passes through a living tissue of the subject 20, and is incident on the light detecting element. In accordance with pulsations of the subject 20, the intensity of the incident light on the light detecting element is changed. Accordingly, changes over time of the incident light intensity may correspond to a waveform of the pulse waves.

In a case where the waveform signal WF is an analog signal, the input interface 111 is provided with an adequate conversion circuit including an A/D converter. This description is similarly applied to other signals that can be received by the input interface 111 and that will be described later.

The information processing device 11 includes a processor 112. The processor 112 is configured to acquire an estimated value of the respiratory rate of the subject 20 based on the waveform signal WF received by the input interface 111.

The information processing device 11 includes an output interface 113. The processor 112 is configured to output, from the output interface 113, a control signal CT that causes the visualizing device 12 to visualize information corresponding to the estimated value of the respiratory rate. The control signal CT may be an analog signal or a digital signal in accordance with the specification of the visualizing device 12.

In other words, the output interface 113 is configured as a hardware interface that can output the control signal CT. In a case where the control signal CT is an analog signal, the output interface 113 provided with an adequate conversion circuit including a D/A converter. This description is similarly applied to other signals that can be outputted from the output interface 113 and that will be described later.

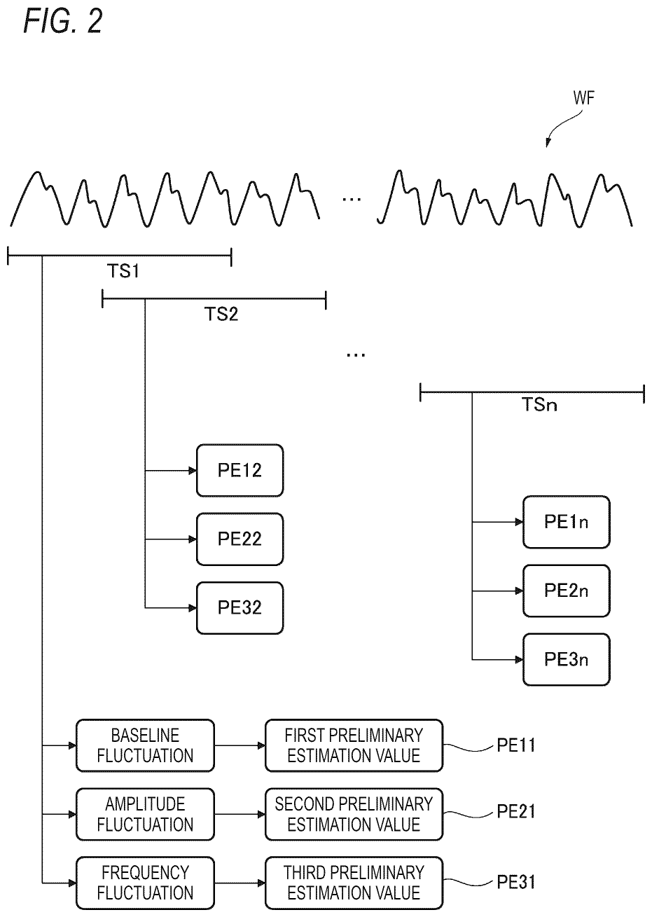

With reference to FIG. 2, a specific flow of processing that is executed by the processor 112 in order to acquire the estimated value of the respiratory rate of the subject 20 will be described.

The waveform of the pulse waves corresponding to the waveform signal WF that is received by the input interface 111 includes a frequency component corresponding to the pulse (0.5 to 2 Hz), a frequency component corresponding to the respiration (0.1 to 1 Hz), and a frequency component of the Mayer wave (0.04 to 0.4 Hz). It is difficult to extract the frequency component corresponding to the respiration only with filtering, because the frequency band thereof partly overlaps with another frequency components.

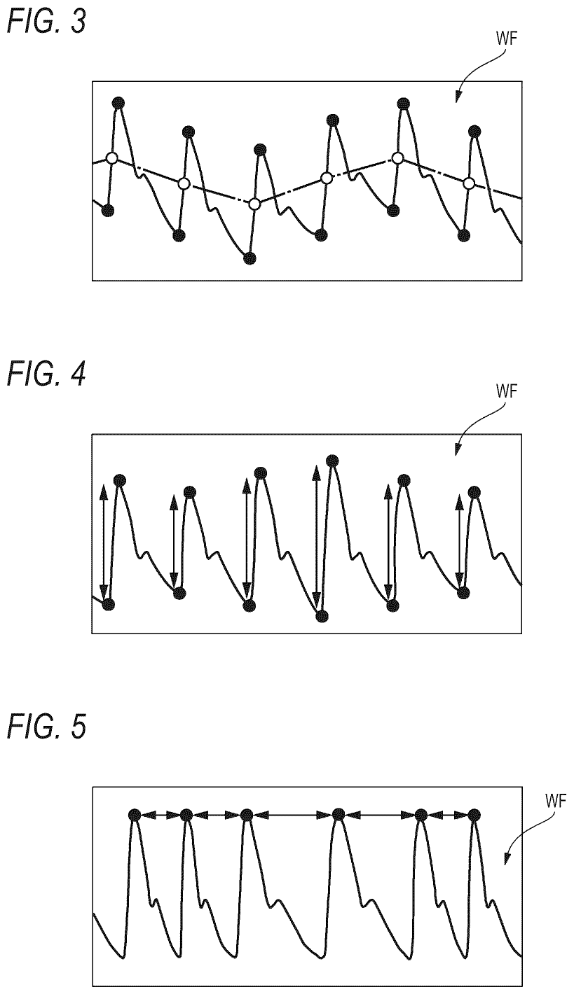

On the other hand, the waveform of the pulse waves includes multiple feature quantities that reflect the influence of respiration. FIG. 3 illustrates a baseline fluctuation of a waveform that is an example of the feature quantity. The baseline fluctuation may be caused by changes in intrathoracic pressure, vasoconstriction of an artery during inspiration, or the like. FIG. 4 illustrates an amplitude fluctuation of a waveform that is another example of the feature quantity. The amplitude fluctuation may be caused by changes in the intrathoracic pressure or the like. FIG. 5 illustrates a frequency fluctuation of a waveform that is another example of the feature quantity. The frequency fluctuation may be caused by a respiratory sinus arrhythmia (RSA), or the like.

Accordingly, as illustrated in FIG. 2, the processor 112 performs processing for extracting the baseline fluctuation, the amplitude fluctuation, and the frequency fluctuation from the waveform signal WF.

Specifically, the processor 112 extracts the multiple feature quantities described above from the waveform signal WF included in a first time slot TS1. The length of the first time slot TS1 is, for example, 30 seconds.

Subsequently, the processor 112 extracts the multiple feature quantities described above from the waveform signal WF included in a second time slot TS2. The length of the second time slot TS2 is equal to the length of the first time slot TS1. An initial point of the second time slot TS2 is later than an initial point of the first time slot TS1. A part of the second time slot TS2 may overlap with the first time slot TS1.

The processor 112 similarly extracts the multiple feature quantities for N time slots. In FIG. 2, a reference symbol TSn represents a N-th time slot.

Subsequently, the processor 112 performs processing for acquiring a preliminary estimation value of the respiratory rate based on each of the multiple feature quantities that are extracted for each of the N time slots. Specifically, by performing frequency analysis processing with respect to each of the feature quantities, a frequency having the largest power spectral value is specified. Examples of the frequency analysis processing include a fast Fourier transform, a wavelet transform, and the like. The processor 112 associates the frequency as specified with a preliminary estimation value of the respiratory rate (the number of respirations per minute).

In this example, the estimated value of the respiratory rate that is acquired based on the baseline fluctuation that is extracted from the first time slot TS1 is referred to as a first preliminary estimation value PE11. Similarly, the estimated value of the respiration rate that is acquired based on the amplitude fluctuation that is extracted from the first time slot TS1 is referred to as a second preliminary estimation value PE21, and the estimated value of the respiration rate that is acquired based on the frequency fluctuation that is extracted from the first time slot TS1 is referred to as a third preliminary estimation value PE31.

Although illustration of the feature quantity is omitted in FIG. 2, the estimated value of the respiratory rate that is acquired based on the baseline fluctuation that is extracted from the second time slot TS2 is referred to as a first preliminary estimation value PE12. Similarly, the estimated value of the respiration rate that is acquired based on the amplitude fluctuation that is extracted from the second time slot TS2 is referred to as a second preliminary estimation value PE22, and the estimated value of the respiration rate that is acquired based on the frequency fluctuation that is extracted from the second time slot TS2 is referred to as a third preliminary estimation value PE32.

Accordingly, the estimated value of the respiratory rate that is acquired based on the baseline fluctuation that is extracted from the N-th time slot TSn is referred to as a first preliminary estimation value PE1n. Similarly, the estimated value of the respiratory rate that is acquired based on the amplitude fluctuation that is extracted from the N-th time slot TSn is referred to as a second preliminary estimation value PE2n, and the estimated value of the respiratory rate that is acquired based on the frequency fluctuation that is extracted from the N-th time slot TSn is referred to as a third preliminary estimation value PE3n.

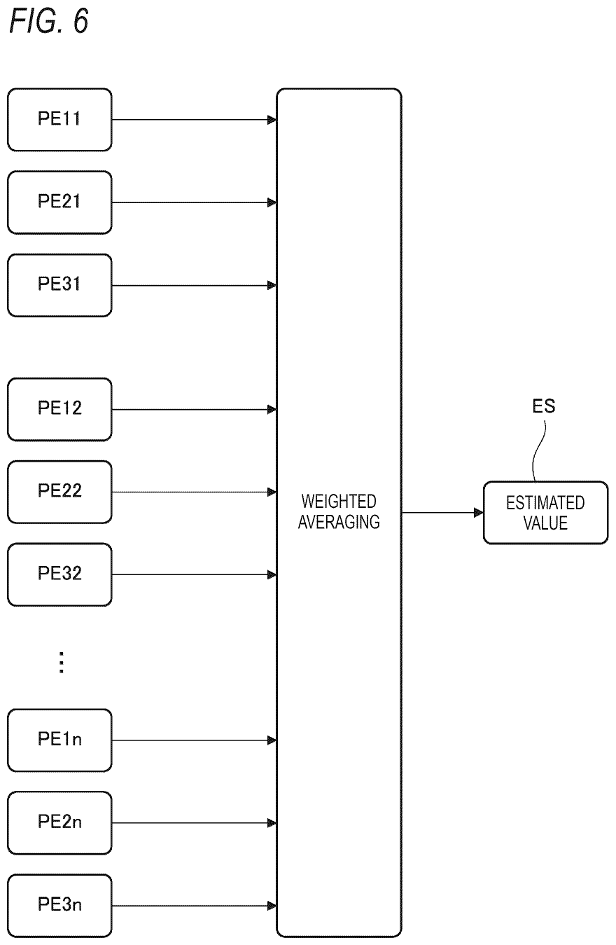

Subsequently, the processor 112 executes weighted averaging for calculating a weighted average value based on the 3N preliminary estimation values that are acquired as described above. FIG. 6 illustrates an exemplary flow of the weighted averaging. The processor 112 determines an estimated value ES of the respiratory rate of the subject 20 to be the weighted average value as specified.

The processor 112 outputs a control signal CT that causes the visualizing device 12 to visualize information corresponding to the calculated estimated value ES of the respiratory rate of the subject 20, from the output interface 113. The information may be an estimation value itself, or a color, a symbol, a figure, or the like corresponding to the estimation value.

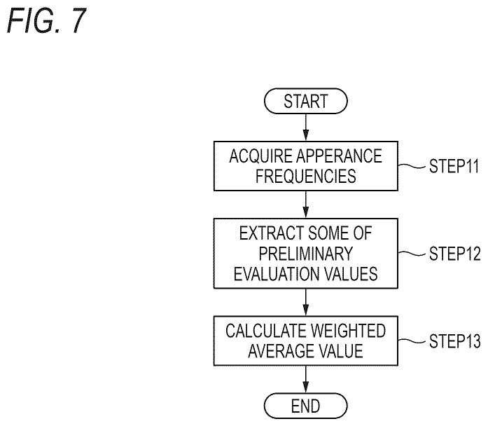

FIG. 7 illustrates a specific flow of the weighted averaging process executed by the processor 112.

In STEP 11, the processor 112 first acquires appearance frequencies of the 3N preliminary estimation values that are acquired as described above.

Subsequently, the processor 112 extracts some of the 3N preliminary estimates (STEP 12). In this example, a preliminary estimation value having the highest appearance frequency, and a preliminary estimation value having the second highest appearance frequency are extracted.

Subsequently, the processor 112 calculates a weighted average value based on the extracted preliminary estimation values and the appearance frequency of each preliminary estimation value (STEP 13). In a case where the highest appearance frequency is f1, the second highest appearance frequency is f2, the preliminary estimation value having the highest appearance frequency is v1, and the preliminary estimation value having the second highest appearance frequency is v2, the weighted average value A is calculated as follows.

$$A = ( f 1 v 1 + f 2 v 2 ) / ( f 1 + f 2 )$$

The inventor of the present application paid attention to a fact that, when the estimated value of the respiratory rate of the subject 20 is determined based on the multiple preliminary estimation values, one or some of them acquired with low accuracy due to some reason may adversely affect the final result of the estimated respiratory rate. According to the configuration of the above-described exemplary processing, the estimation value ES of the respiratory rate of the subject 20 is determined based on the weighted average value A that is calculated such that the influence of the preliminary estimation value having higher appearance frequency is enhanced. Accordingly, it is possible to suppress affection of a preliminary estimation value having lower appearance frequency that would possibly be an abnormal value. Since such processing is executed by the processor 112 installed in the information processing device 11, it is possible to suppress the deterioration in the estimation accuracy of the respiratory rate of the subject 20 without increasing the number of sensors.

As a measure for reducing the operation load of the processor 112, it is conceivable to shorten the time slot that is to be applied to the waveform signal WF. However, the shortening of the time slot causes the resolution of the frequency analysis to be lowered. Particularly in this example, since the weighted average value A is calculated by using a lesser number of preliminary estimation values that are extracted in descending order of the appearance frequencies, it is possible to acquire a value that cannot be obtained by the initial resolution. In other words, it is possible to solve the problem of insufficiency of the resolution. Accordingly, it is possible to suppress deterioration of the estimation accuracy of the respiratory rate of the subject 20 even though the increase in the operation load of the processor 112 is suppressed by shortening the time slot.

However, the weighted average value A may be calculated based on three or more preliminary estimation values having different appearance frequencies.

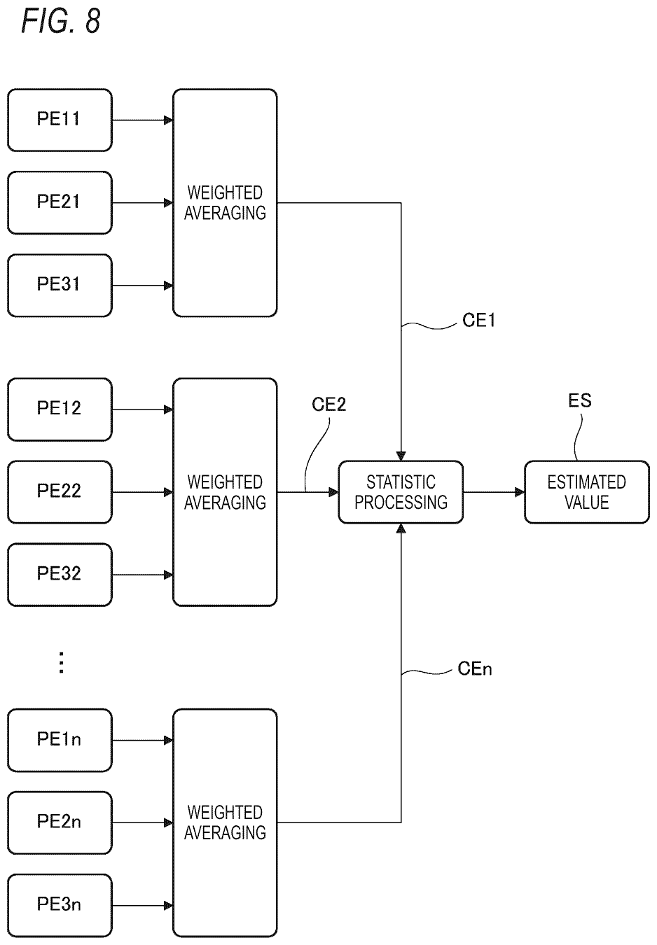

FIG. 8 illustrates another exemplary weighted averaging executed by the processor 112. In this example, the weighted averaging is first performed with respect to the multiple preliminary estimation values that are acquired in each of the N time slots.

Specifically, a weighted average value is calculated based on the appearance frequencies of the first preliminary estimation value PE11, the second preliminary estimation value PE21, and the third preliminary estimation value PE31 that are acquired based on the multiple feature quantities that are extracted in the first time slot TS1. The weighted average value is handled as a first candidate estimation value CE1.

Similarly, a weighted average value is calculated based on the appearance frequencies of the first preliminary estimation value PE12, the second preliminary estimation value PE22, and the third preliminary estimation value PE32 that are acquired based on the multiple feature quantities that are extracted in the second time slot TS2. The weighted average value is handled as a second candidate estimation value CE2.

The above processing is performed with respect to all N time slots, so that a weighted average value is calculated based on the appearance frequencies of the first preliminary estimation value PE1n, the second preliminary estimation value PE2n, and the third preliminary estimation value PE3n that are acquired based on the multiple feature quantities extracted in the N-th time slot TSn. The weighted average value is handled as an n-th candidate estimation value CEn.

Subsequently, the processor 112 executes statistic processing for specifying representative values of the N candidate estimation values that are acquired as described above to determine an estimated value ES of the respiratory rate of the subject 20 to be the representative value as specified. Examples of the representative value include a mean value, a median value, and a mode value.

The estimation accuracy of the respiratory rate would be deteriorated due to temporary noise or the like that is generated during the acquisition of the waveform signal WF. According to the above-described exemplary processing, since the weighted average value that is calculated based on the appearance frequencies of the multiple preliminary estimation values acquired in each of the multiple time slots is specified as the candidate estimation value, so that the estimated value ES of the respiration rate of the subject 20 is determined to be the representative value of the multiple candidate estimation values. Accordingly, suppression of the affection of temporal cause to the estimation accuracy deterioration can be facilitated. Accordingly, it is possible to suppress the deterioration of the estimation accuracy of the respiratory rate of the subject 20 without increasing the number of sensors.

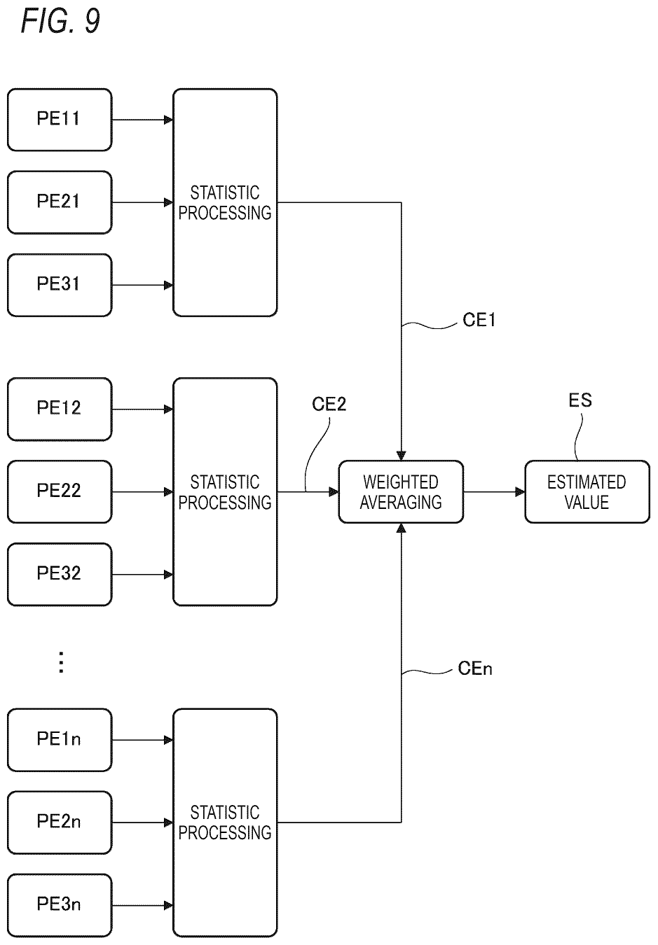

As illustrated in FIG. 9, the above-described statistic processing may be first performed with respect to the multiple preliminary estimation values that are acquired in each of the N time slots, whereby N candidate estimation values CE1 to CEn are acquired. In this case, the estimated value ES of the respiratory rate of the subject 20 is determined, by the weighted averaging, to be a weighted average value based on the appearance frequencies of the N candidate estimated values CE1 to CEn.

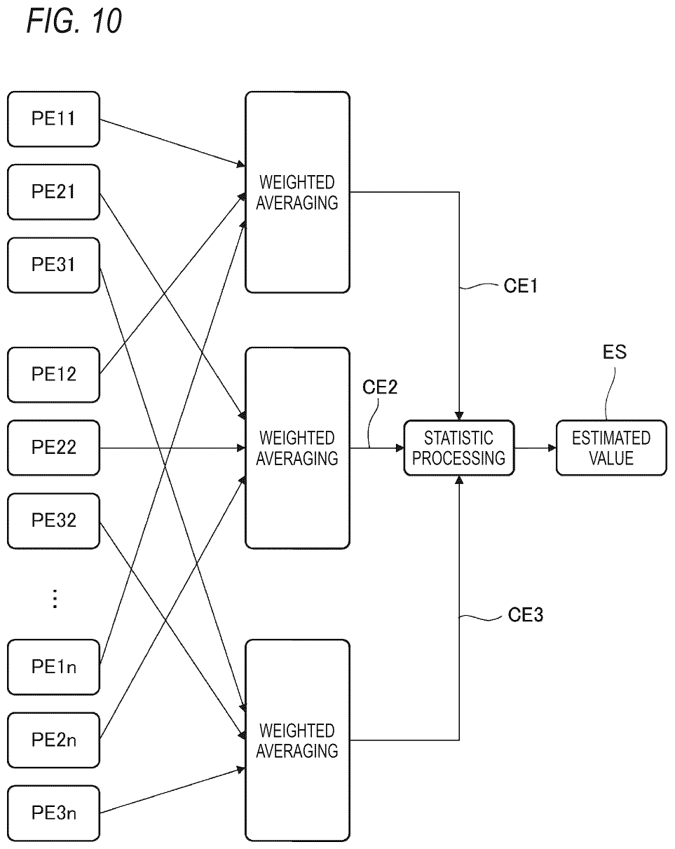

FIG. 10 illustrates another exemplary weighted averaging executed by the processor 112. In this example, weighted averaging is first performed with respect to the N preliminary estimation values that are acquired from the N time slots for each of the three feature quantities that are extracted from the waveform signal WF.

Specifically, a weighted average value is calculated based on the appearance frequencies of N first preliminary estimation values including the first preliminary estimation value PE11, the first preliminary estimation value PE12, . . . , and the first preliminary estimation value PE1n that are acquired based on the baseline fluctuation extracted from the waveform signal WF. The weighted average value is handled as a first candidate estimation value CE1. Similarly, a weighted average value is calculated based on the appearance frequencies

of the N second preliminary estimation values including the second preliminary estimation value PE21, the second preliminary estimation value PE22, . . . , and the second preliminary estimation value PE2n that are acquired based on the amplitude fluctuation extracted from the waveform signal WF. The weighted average value is handled as a second candidate estimation value CE2.

Similarly, a weighted average value is calculated based on the appearance frequencies of the N third preliminary estimation values including the third preliminary estimation value PE31, the third preliminary estimation value PE32, . . . the third preliminary estimation value PE 3n that are acquired based on the frequency fluctuation extracted from the waveform signal WF. The weighted average value is handled as a third candidate estimation value CE3.

Subsequently, the processor 112 executes statistic processing for specifying representative values of the three candidate estimation values that are acquired as described above to determine an estimated value ES of the respiratory rate of the subject 20 to be the representative value as specified. Examples of the representative value include a mean value, a median value, and a mode value.

The state of the subject may deteriorate the estimation accuracy of the respiration rate based on a particular feature quantity. As an example, in a case where the respiration frequency of the subject is low (e.g., 10 or less per minute), a tendency has been found that the estimation accuracy of the respiration rate based on the baseline fluctuation is low. As another example, there has been found a tendency that the estimation accuracy of the respiration rate based on the frequency fluctuation is deteriorated in a case where an age (particularly a vascular age) of the subject is high.

According to the above-described exemplary processing, a weighted average value calculated based on the appearance frequencies of the multiple preliminary estimation values that are acquired in each of the multiple feature quantities is specified as a candidate estimation value, so that an estimated value ES of the respiratory rate of the subject 20 is determined to be a representative value of the multiple candidate estimation values. Accordingly, suppression of the affection of cause related to the particular feature amount to the estimation accuracy deterioration can be facilitated. Accordingly, it is possible to suppress the deterioration of the estimation accuracy of the respiratory rate of the subject 20 without increasing the number of sensors.

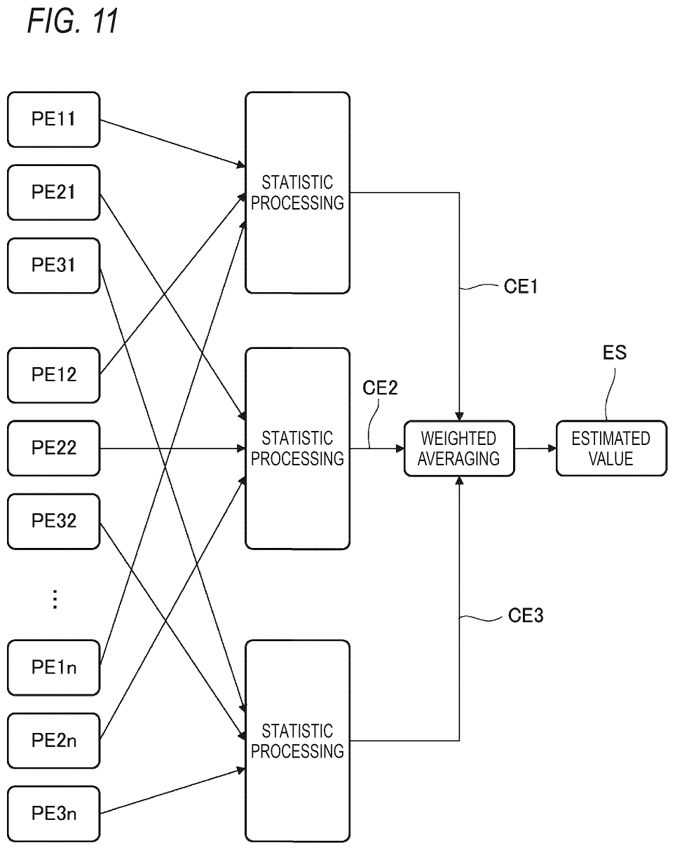

It should be noted that, as illustrated in FIG. 11, the above-described statistic processing may be first performed with respect to the N preliminary estimation values that are acquired from the N time slots for each of the three feature quantities extracted from the waveform signal WF, whereby three candidate estimation values CE1 to CE3 are acquired. In this case, the estimated value ES of the respiratory rate of the subject 20 is determined, by the weighted averaging, to be a weighted average value based on the appearance frequencies of the three candidate estimated values CE1 to CE3.

The processor 112 of the information processing device 11 having various functions as exemplified above may be implemented by at least one non-exclusive microprocessor configured to cooperate with at least one non-exclusive memory. Examples of the general-purpose microprocessor include a CPU, an MPU, and a GPU. Examples of the general-purpose memory include a ROM, a RAM, and the like. In this case, a computer program that implements the various functions described above may be stored in the ROM. The ROM is an example of a non-transitory computer-readable medium having stored a computer program. The non-exclusive microprocessor designates at least a part of the program stored in the ROM, loads the designated program in the RAM, and executes the above-described processing in cooperation with the RAM. The computer program may be pre-installed in a non-exclusive memory, or may be downloaded from an external server device with a communication network, and then installed in the non-exclusive memory. In this case, the external server device is an example of the non-transitory computer-readable medium having stored the computer program.

The processor 112 may be implemented by at least one exclusive integrated circuitry capable of executing the above-described computer program. Examples of the dedicated integrated circuit include a microcontroller, an ASIC, and an FPGA. In this case, the above-described computer program is pre-installed in the memory element included in the dedicated integrated circuit. The memory element is an example of a computer-readable medium having stored therein a computer program. The processor 112 may also be implemented by a combination of the non-exclusive microprocessor and the exclusive integrated circuitry.

The various configurations described above are merely illustrative for facilitating understanding of the presently disclosed subject matter. Each of the illustrative configurations may be appropriately modified or combined with another illustrative configuration within the gist of the present disclosure.

In the above exemplary embodiment, three feature quantities including the baseline fluctuation, the amplitude fluctuation, and the frequency fluctuation are extracted from the waveform signal WF. According to the above configuration, since a larger number of preliminary estimation values can be acquired based on various feature quantities, it is easy to suppress the influence of various causes that degrade the estimation accuracy of the respiratory rate. However, as long as a sufficient number of preliminary estimation values can be acquired, the feature quantity that is to be acquired from the waveform signal WF may be at least one.

In the above exemplary embodiment, the estimated value ES of the respiratory rate of the subject 20 is visualized by the visualizing device 12. However, the estimation value ES may be outputted as sound by a speaker, or may be transmitted to another apparatus as data by a data transmitting apparatus. In this case, the speaker or the data transmitting device may be an example of the output device.

In the above exemplary embodiment, a waveform signal WF corresponding to the waveform of the pulse wave of the subject 20 is acquired. However, a waveform signal WF corresponding to the waveform of the electrocardiogram of the subject 20 may be acquired. Namely, the electrocardiogram may be an example of physiological information involving a periodic change.

1. An information processing device configured to process physiological information of a subject, comprising:
an interface configured to receive a signal corresponding to a waveform of physiological information involving cyclic changes from a sensor attached to the subject; and
a processor configured to cause, in response to the signal, an output device to output information corresponding to an estimated value of a respiration rate of the subject,
wherein the processor is configured to:
extract multiple feature quantities corresponding to respiration of the subject from the signal included in each of multiple time slots;
apply frequency analysis processing with respect to each of the feature quantities to acquire multiple preliminary estimation values of the respiration rate; and
determine the estimated value based on a weighted average value that is associated with appearance frequencies of the preliminary estimation values.

2. The information processing device according to claim 1,
wherein the processor is configured to calculate the weighted average value based on a lesser number of preliminary evaluation values that are extracted from the preliminary evaluation values.

3. The information processing device according to claim 1,
wherein the processor is configured to:
acquire multiple candidate estimation values by calculating the weighted average value of the preliminary estimation values of the feature quantities that are acquired in each of the time slots; and
determine the estimated value to be a representative value of the candidate estimation values.

4. The information processing device according to claim 1,
wherein the processor is configured to:
acquire multiple candidate estimation values by calculating the weighted average value of the preliminary estimation values that are acquired in the time slots for each of the feature quantities; and
determine the estimated value to be a representative value of the candidate estimation values.

5. The information processing device according to claim 1,
wherein the feature quantities include a baseline fluctuation, an amplitude fluctuation, and a frequency fluctuation.

6. The information processing device according to claim 1,
wherein the physiological information includes pulse waves.

7. A monitoring device configured to monitor physiological information of a subject, comprising:
an interface configured to receive a signal corresponding to a waveform of physiological information involving cyclic changes from a sensor attached to the subject;
an output device; and
a processor configured to cause, in response to the signal, the output device to output information corresponding to an estimated value of a respiration rate of the subject,
wherein the processor is configured to:
extract multiple feature quantities corresponding to respiration of the subject from the signal included in each of multiple time slots;
apply frequency analysis processing with respect to each of the feature quantities to acquire multiple preliminary estimation values of the respiration rate; and
determine the estimated value based on a weighted average value that is associated with appearance frequencies of the preliminary estimation values.

8. A non-transitory computer-readable medium having stored a computer program adapted to be executed by a processor installed in an information processing device configured to process physiological information of a subject, the computer program being configured to, when executed, cause the information processing device to:
receive a signal corresponding to a waveform of physiological information involving cyclic changes from a sensor attached to the subject;
extract multiple feature quantities corresponding to respiration of the subject from the signal included in each of multiple time slots;
apply frequency analysis processing with respect to each of the feature quantities to acquire multiple preliminary estimation values of a respiration rate of the subject;
determine an estimated value of the respiration rate based on a weighted average value that is associated with appearance frequencies of the preliminary estimation values; and
cause an output device to output information corresponding to the estimated value.
